(19) United States
(12) Patent Application Publication
(10) Pub. No.: US 2003/0088195 A1
(43) Pub. Date: May 8, 2003

(54) Guidewire having measurement indicia

(76) Inventors: Gil M. Vardi, Town and Country, MO (US); Michael Hogendijk, Palo Alto, CA (US)

Correspondence Address: FISH & NEAVE, 1251 AVENUE OF THE AMERICAS, 50TH FLOOR, NEW YORK, NY, 10020-1105, US

(21) Appl. No.: 10/052,808

(22) Filed: Nov. 2, 2001

(51) Int. Cl. A61B 5/00

(52) U.S. Cl. 600/585

(57) ABSTRACT

Apparatus and methods for manufacturing a guidewire having a plurality of radiopaque markers are disclosed. In a preferred embodiment, the present invention provides a guidewire having a tapered distal section comprising a plurality of gold markers that are deposited on the guidewire at predetermined intervals, so that the outer surface of the guidewire is substantially smooth. The gold markers provide a fluoroscopic reference for positioning the guidewire and enable accurate sizing of vessel features, such as the length of a lesion.

### FIELD OF THE INVENTION

The present invention relates to improved apparatus for measuring features of vessels. More specifically, the present invention provides a guidewire having a plurality of radiopaque markers useful for sizing the length of a lesion, e.g., within coronary arteries.

### BACKGROUND OF THE INVENTION

Patients suffering from atherosclerosis may undergo angioplasty, a procedure involving the use of a balloon-tipped catheter that dilates occluded vessels by compressing atherosclerotic plaque against the vessel wall. Further benefits may be realized if the patient additionally undergoes stenting, a process involving the deployment of tubular prostheses that hold the occluded vessel open and help restore adequate blood flow to the region.

Guidewires having relatively small diameters and flexible coiled tips may be used to transluminally navigate the tortuous anatomy and locate lesions prior to insertion of the catheter. Additionally, guidewires may be used to size vascular lesions prior to performing interventional procedures to determine the size of the angioplasty balloon or stent to be used in treating the lesion. Accurately assessing the three-dimensional size of a lesion requires a physician to account for lesions that partially extend into a third dimension not visible on a two-dimensional fluoroscopy screen.

Several previously-known guidewires have been introduced for use in positioning balloon catheters within a vessel and/or sizing vessel characteristics. U.S. Pat. No. 5,174,302 to Palmer describes a guidewire having an initially uniform core section that tapers inward along a distal segment. The distal segment is surrounded by a flexible spring tip that is banded to define portions that are highly radiopaque and portions that are much less radiopaque. The radiopaque bands provide a reference for the physician with regard to positioning the guidewire within the cardiovascular system when used in conjunction with an x-ray imaging system.

The previously known device described in the foregoing patent has several drawbacks. First, despite having a tapered distal segment of core wire, the overall diameter of the guidewire is substantially equal along its length because the radiopaque-banded spring that wraps around the distal segment adds to the core wire diameter and negates the tapering effect. It therefore would be beneficial to provide a guidewire having a reduced distal diameter that facilitates use in smaller vessels.

Another drawback associated with the device described in the Palmer patent is that the radiopaque markers are disposed in the coiled spring. While it may be desirable to simultaneously provide the radiopaque guidewire within the stenosis, e.g., as a reference point throughout a stenting procedure, the Palmer device may be difficult to track in real time under fluoroscopy. This is because a distal coil is typically advanced through and disposed distal to the stenosis, not disposed within the stenosis itself, which may make the coil difficult to view throughout the procedure.

Cook Incorporated offers a measuring guidewire under the tradename GRADUATE®, for use in sizing vessel lumens prior to angioplasty and other interventional procedures. This product has six distal gold markers spaced 1 cm apart and four proximal markers spaced at 5 cm intervals disposed on the distal end of the guidewire.

One drawback associated with the Cook guidewire is its relatively large diameter. The guidewire diameter is 0.035 inches, and therefore is not suitable for use in coronary arteries. Additionally, the gold marker bands are affixed to the outer diameter of the guidewire, and result in an increased diameter that forms a potentially uneven surface.

In view of these drawbacks of previously known guidewires, it would be desirable to provide a guidewire having radiopaque markers suitable for accurately sizing the length of a feature, e.g., a lesion, within a vessel.

It also would be desirable to provide a guidewire having radiopaque markers that is suitable for insertion into smaller vessels, e.g., coronary arteries.

It still further would be desirable to provide a guidewire having radiopaque markers that form a substantially smooth surface along the guidewire such that the bands do not increase the diameter of the guidewire or create a jagged surface.

### SUMMARY OF THE INVENTION

In view of the foregoing, it is an object of the present invention to provide a guidewire having radiopaque markers suitable for accurately sizing the length of a lesion within a vessel.

It is also an object of the present invention to provide a guidewire having a plurality of radiopaque markers that is suitable for insertion into smaller vessels, e.g., coronary arteries.

It is further an object of the present invention to provide a guidewire having a plurality of radiopaque markers that form a substantially smooth surface along the guidewire such that the bands do not increase the diameter of the guidewire or create an uneven surface.

These and other objects of the present invention are accomplished by providing a guidewire having proximal and distal sections, and a plurality of radiopaque markers disposed along the distal section at predetermined intervals. The markers may be evenly spaced, for example, 10 mm apart, to enable a physician to accurately assess the size of a vessel feature, such as a lesion.

In a preferred embodiment, the guidewire comprises a core wire having a constant diameter proximal section and a tapered distal section having a plurality of radiopaque marker bands, preferably inset into indentations formed in the outer surface of the core wire to provide a substantially smooth surface. The guidewire also may include a lubricious surface, such as polytetrafluoroethelene (“PTFE”) disposed on its outer surface.

The guidewire of the present invention is manufactured by first masking the tapered distal section of core wire. The desired locations for the radiopaque markers then are selected, and the mask removed from the core wire at those selected locations to expose the core wire, e.g., by mechanically abrading or chemically removing the masking. A radiopaque material, preferably gold, then is deposited, such as by electroplating or vacuum deposition, on the distal section so that the selected, exposed regions of core wire are coated, while the mask prevents coating of other regions of the core wire. The mask then is removed.

Preferably, small indentations may be provided in the core wife, e.g., by grinding or chemically etching the core wire prior to deposition of the radiopaque material, so that no additional diameter is added to the guidewire. A lubricious coating then may be applied to further ensure a smooth, nonstick surface.

In an alternative embodiment, a sheath having a plurality of radiopaque markers disposed at predetermined intervals along its distal section may be used in combination with a traditional guidewire. In this embodiment, the traditional guidewire navigates the tortuous vasculature and crosses the lesion, then the sheath is distally advanced over the guidewire and the length of the lesion is assessed using the radiopaque markers of the sheath.

### BRIEF DESCRIPTION OF THE DRAWINGS

Further features of the invention, its nature and various advantages will be more apparent from the accompanying drawings and the following detailed description of the preferred embodiments, in which:

FIG. 1 illustrates a guidewire constructed in accordance with the principles of the present invention;

FIG. 2 illustrates the apparatus of the present invention positioned within an occluded vessel;

FIG. 3 describe a method of manufacturing apparatus in accordance with the present invention; and

FIG. 4 describe an alternative embodiment for measuring features of a vessel using a sheath having a plurality of radiopaque markers; and

FIG. 5 describe an alternative embodiment for measuring features of a vessel using a sheath in a rapid-exchange manner.

### DETAILED DESCRIPTION OF THE INVENTION

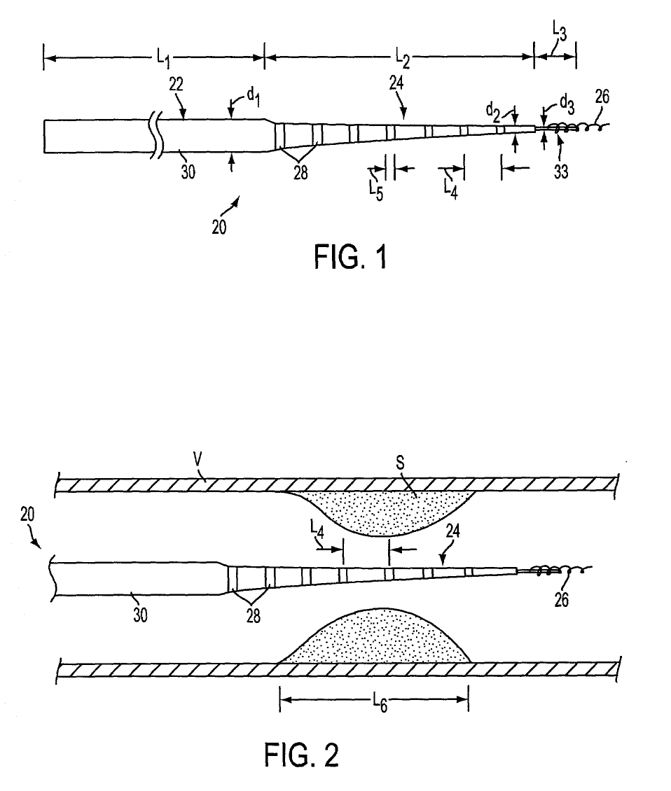

Referring to FIG. 1, guidewire 20 constructed in accordance with principles of the present invention is described. Guidewire 20 comprises core wire 30 having proximal section 22, distal section 24, and optionally, reduced diameter distalmost section 33.

Along proximal section 22, core wire 30 spans a length L₁ that comprises the majority of the overall length of guidewire 20. Proximal section 22 may comprise a constant diameter d₁, preferably 0.014 inches. Along distal section 24, core wire 30 may taper inward gradually over a length L₂. In a preferred embodiment, L₂ spans approximately 30 cm, and core wire 30 tapers uniformly to a final diameter d₂, preferably about 0.005 inches.

Distal section 24 comprises plurality of radiopaque markers 28. In a preferred embodiment, radiopaque markers 28 are spaced at equal intervals L₄, for example, spaced apart 10 mm center-to-center, and the markers are 1 mm in length, as represented by length L₅. Radiopaque markers 28 preferably consist of a gold layer that is electroplated or otherwise deposited onto core wire 30 according to manufacturing techniques described hereinbelow.

Guidewire 20 may further comprise a reduced diameter, distalmost section 33 having core wire diameter d₃. Core wire diameter d₃ preferably is between about 0.001 and 0.003 inches. Coil 26 may be affixed to distalmost section 33, such that the added diameter of coil 26 to reduced core wire diameter d₃ does not substantially increase the overall diameter of section 33 relative to diameter d₂.

Referring to FIG. 2, guidewire 20 constructed in accordance with the present invention is depicted within a vessel V, for example, a coronary artery, having a lesion S that spans a length L₆. Radiopaque markers 28 of guidewire 20 may be used to measure the length of lesion S under fluoroscopy since the markers are spaced at known, and preferably equal, intervals. For example, the appearance of four radiopaque markers 28 along the length of lesion S may translate into a lesion that is approximately 40 mm in length, assuming that markers 28 are equally spaced at 10 mm intervals, center-to-center.

Advantageously, several radiopaque markers may be provided along distal section 24 to better assess the characteristics of vessel V. The number of radiopaque markers is dependent on the overall length of tapered distal section 24 and the spacing intervals, L₄. In a preferred embodiment, when tapered distal section 24 spans 30 cm and radiopaque markers 28 are equally spaced 10 mm apart, core wire 30 may accommodate approximately 30 markers.

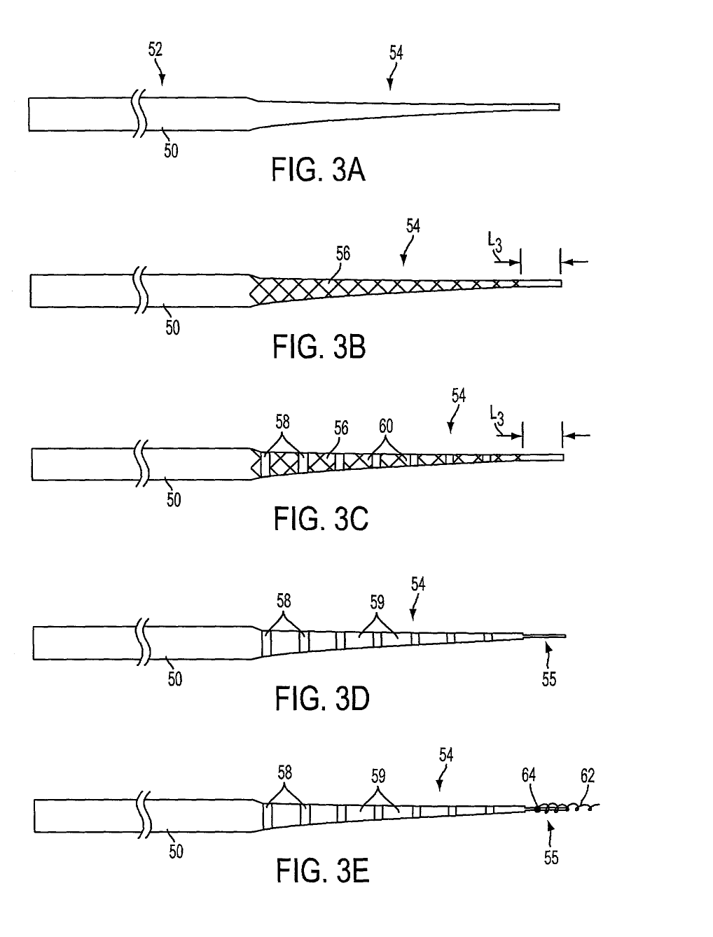

Referring to FIG. 3, a method of manufacturing apparatus in accordance with principles of the present invention is described. Guidewire 20 comprises a length of core wire 50 having an initially constant diameter along proximal section 52 and distal section 54. In a preferred embodiment, the initial diameter of core wire 50 is 0.014 inches. Distal section 54 of core wire 50 then is taper-ground such that its diameter gradually decreases, as shown in FIG. 3A. The taper preferably spans the distal 30 cm of core wire 50 and tapers from 0.014 to 0.005 inches.

Distal section 54 of core wire 50 then is coated using a masking 56, for example, FEP (Fluorinated Ethylene-Propylene), silicone rubber, paint or another method, as shown in FIG. 3B. A distalmost section L₃ may be set aside, i.e., neither tapered nor masked, for the purpose of subsequently adhering a coil to the distal end of the guidewire.

Once distal section 54 is masked, the desired locations for the radiopaque markers may be selected. Mask 56 then is removed primarily at the selected locations, e.g., by scraping, abrading or chemically removing the mask at the selected locations, such that distal section 54 comprises exposed regions 58 and masked regions 60, as shown in FIG. 3C. The dimensions and locations of exposed regions 58 are selected based on the desired positioning of the radiopaque markers, and are preferably 1 mm in length and spaced 10 mm apart, center-to-center.

A radiopaque material, preferably gold, then is deposited on distal section 54 at exposed regions 58, for example, by electroplating or vacuum deposition, while masked regions 60 prevent coating of unwanted regions of core wire 50. More preferably, exposed regions 58 may be reduced in diameter, e.g. by grinding or chemically etching, to form indentations prior to deposition of the radiopaque material. In this manner, the finished guidewire will have a substantially smooth outer surface, with the radiopaque markers substantially flush with the outer diameter of the core wire.

The remaining mask that covers the masked regions 60 then may be removed, either by use of dissolving chemicals or scraping the layer of masking. Upon removal of the remaining masking, distal section 54 of guidewire 20 comprises radiopaque markers 58 and non-radiopaque regions 59 of core wire 50, as shown in FIG. 3D.

Distalmost section L₃ then may be flattened to form reduced diameter distalmost section 55, as shown in FIG. 3D. The diameter of distalmost section 55 preferably is between about 0.001 and 0.003 inches. The reduced core wire diameter along distalmost section 55 allows coil 62 to be affixed to core wire 50 such that it does not substantially increase the diameter relative to the diameter provided at the distal end of section 54, which is preferably 0.005 inches. Adhesive 64, e.g., a solder or weld, may be used to affix coil 62 to section 55 of core wire 50, as shown in FIG. 3E.

Coil 62 is configured to transluminally guide apparatus 20 through tortuous vasculature and into the selected vessel. Coil 62 preferably comprises a radiopaque material, e.g., platinum, to facilitate fluoroscopic guidance of the device. Coil 26 may overlap exclusively with section 55 of core wire 50, or may extend distally beyond core wire 50. Alternatively, reduced diameter distalmost section 55 may be omitted and coil 62 may be affixed directly to the distal end of section 54.

A lubricious coating, preferably, e.g., polytetrafluoroethylene (“PTFE”) is applied to core wire 50 to ensure a smooth surface suitable for vascular insertion.

Although the marker bands of the present invention are illustratively depicted as circumferential bands, one of ordinary skill in the art will recognize that the sizes and shapes of the radiopaque markers may vary. For example, the radiopaque markers may comprise rectangular shapes, circular shapes, or irregular banded shapes that extend circumferentially around core wire.

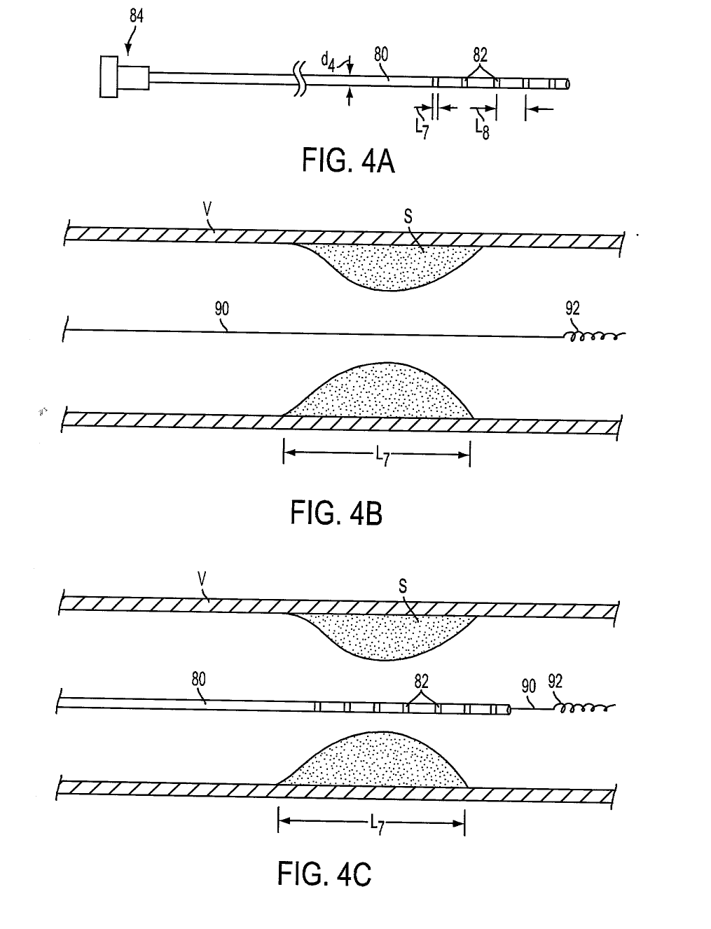

Referring to FIG. 4, alternative apparatus and methods for measuring features of vessels are described. In FIG. 4A, sheath 80 having proximal and distal sections comprises plurality of radiopaque markers 82 disposed at predetermined intervals along the distal section. In this embodiment, the proximal end of sheath 80 communicate with proximal hub 84. In a preferred embodiment, radiopaque markers 82 are spaced at equal intervals L₈, for example, spaced apart 10 mm center-to-center, and the markers are 1 mm in length, as represented by L₇. Radiopaque markers 82 preferably consist of a gold layer that is electroplated or otherwise deposited onto sheath 80, according to manufacturing techniques described in FIGS. 3B-3D hereinabove. Using such techniques, sheath 80 will have a substantially smooth outer surface, with radiopaque markers 82 being substantially flush with outer diameter d₄ of sheath 80. Sheath 80 preferably comprises a material used in catheter construction, such as polyethylene or polyimide, and has a wall thickness of about 0.001 to 0.005 inches.

Sheath 80 of FIG. 4A is used in combination with a previously known guidewire having proximal and distal ends to measure features of a vessel. In a first method step, the distal end of traditional guidewire 90 is transluminally inserted into occluded vessel V. The distal end of traditional guidewire 90 preferably crosses lesion S and is ultimately positioned distal to lesion S, as shown in FIG. 4B. The distal end of traditional guidewire 90 preferably comprises coil 92 configured to transluminally navigate tortuous vasculature.

Sheath 80, having an inner diameter slightly larger than the outer diameter of guidewire 90, then is distally advanced over guidewire 90 and positioned within lesion S, as shown in FIG. 4C. Radiopaque markers 82 may be used to measure the length of lesion S under fluoroscopy since the markers are spaced at known, and preferably equal, intervals. Radiopaque markers 82 allow a physician to accurately assess L₇, even though lesion S may partially extend into a third dimension not visible under two-dimensional fluoroscopy. Upon sizing L₇, sheath 80 may be removed from the patient's body and an appropriately-sized angioplasty balloon catheter or stent may be delivered to the site of the lesion via guidewire 90.

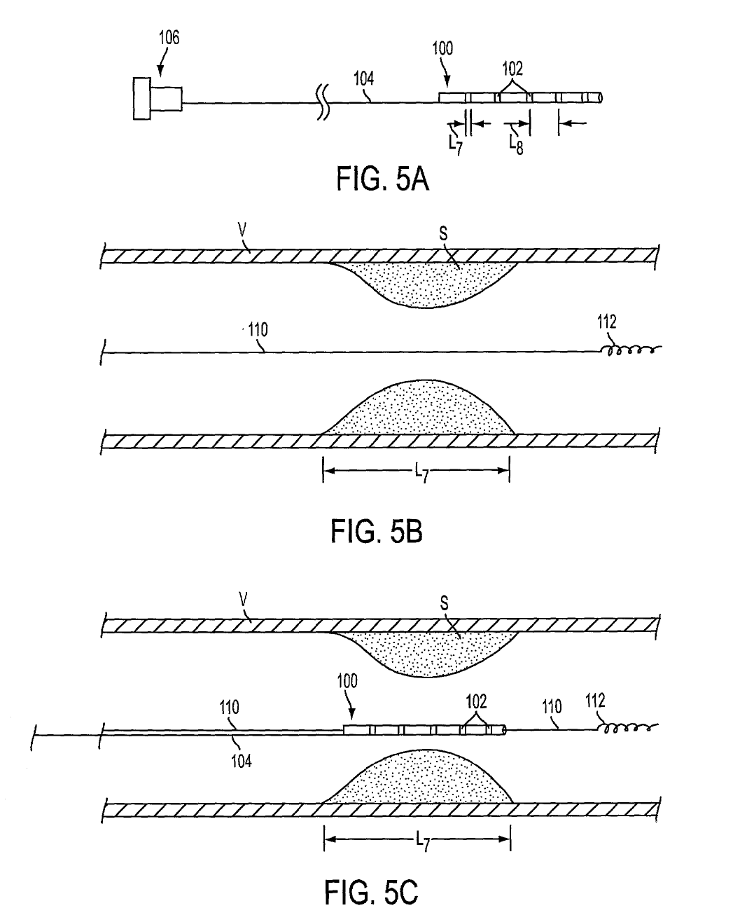

Referring to FIG. 5, apparatus and methods suitable for using a measuring sheath in a rapid-exchange manner are described. In FIG. 5A, sheath 100 comprises plurality of radiopaque markers 102 disposed at predetermined intervals along its length. Sheath 100 and radiopaque markers 102 are provided in accordance with manufacturing techniques described hereinabove. Sheath 100 is coupled to push wire 104, e.g., a stainless steel wire or shaft having an outer diameter of about 0.014 inches, that is suitable for transmitting forces to sheath 100. Push wire 104 preferably spans a substantially greater length than sheath 100, and the proximal end of push wire 104 communicates with proximal hub 106.

Sheath 100 of FIG. 5A may used in combination with a previously known guidewire having proximal and distal ends to measure features of a vessel. In a first method step, the distal end of traditional guidewire 110 is transluminally inserted into occluded vessel V. The distal end of traditional guidewire 110 preferably crosses lesion S and is ultimately positioned distal to lesion S, as shown in FIG. 4B.

Sheath 100, having an inner diameter slightly larger than the outer diameter of guidewire 110, is positioned over the proximal end of guidewire 110. Push wire 104 then is advanced distally and causes sheath 100 to translate distally. Sheath 100 is ultimately positioned within lesion S, as shown in FIG. 4C, and radiopaque markers 102 may be used to measure the length of lesion S under fluoroscopy. The use of push wire 104 advantageously permits guidewire 110 to have a relatively small length, i.e., spanning approximately from the site of the lesion to a location just outside of the patient's body, such that the apparatus may be used in a rapid-exchange manner. Push wire 104 then may be retracted proximally to remove sheath 100 from the patient's body upon completion of the step of measuring the vascular feature.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. A guidewire suitable for measuring features within a vessel, the guidewire comprising a core wire having proximal and distal sections, the distal section having an outer surface and outer diameter, and a plurality of radiopaque markers disposed at predetermined intervals on the distal section, the improvement comprising that the outer surface of the guidewire is substantially smooth.

2. The guidewire of claim 1 wherein the plurality of radiopaque markers are substantially flush with the outer diameter of the distal section.

3. The guidewire of claim 2 wherein the diameter of the proximal section is about 0.014 inches.

4. The guidewire of claim 1 wherein the distal section of the core tapered.

5. The guidewire of claim 4 wherein the distal section tapers gradually from 0.014 inches to about 0.005 inches.

6. The guidewire of claim 1 wherein the plurality of radiopaque markers are each about 1 mm in length.

7. The guidewire of claim 1 wherein the plurality of radiopaque markers are disposed at equally spaced intervals.

8. The guidewire of claim 7 wherein the equally spaced intervals are about 10 mm.

9. The guidewire of claim 1 wherein the plurality of radiopaque markers are deposited into indentations in the outer surface of the core wire.

10. The guidewire of claim 1 further comprising a lubricious coating covering the core wire.

11. The guidewire of claim 1 wherein the distal 30 mm of the core wire has a reduced diameter between about 0.001 and 0.003 inches.

12. The guidewire of claim 11 further comprising a coil having proximal and distal ends, the proximal end of the coil affixed to the distal 30 mm of core wire.

13. The guidewire of claim 12 wherein the coil consists of platinum.

14. The guidewire of claim 1 wherein the radiopaque markers consist of gold.

15. A method for manufacturing a guidewire suitable for measuring features within a vessel, the method comprising:
providing a core wire having proximal and distal sections;
coating portions of the distal section with a mask;
exposing the core wire beneath the mask at a plurality of predetermined locations;
depositing a radiopaque material at the predetermined locations; and
removing the mask.

16. The method of claim 15 further comprising tapering the distal section of the core wire.

17. The method of claim 15 wherein depositing a radiopaque material at the predetermined locations comprises electroplating gold onto the predetermined locations.

18. The method of claim 15 further comprising coating the guidewire with a lubricious coating.

19. The method of claim 15 wherein removing the mask includes chemically dissolving the masking.

20. The method of claim 15 further comprising forming indentations at the predetermined locations prior to depositing the radiopaque materials.

21. Apparatus suitable for measuring features within a vessel, the apparatus comprising:
a sheath having a distal region, the distal region having an exterior surface and outer diameter, and a plurality of radiopaque markers disposed at predetermined intervals on the exterior surface.

22. The apparatus of claim 21 wherein the plurality of radiopaque markers are substantially flush with the outer diameter of the distal region.

23. The apparatus of claim 21 wherein the plurality of radiopaque markers are each about 1 mm in length.

24. The apparatus of claim 21 wherein the plurality of radiopaque markers are disposed spaced apart at equally spaced intervals.

25. The apparatus of claim 24 wherein the equally spaced intervals are about 10 mm.

26. The apparatus of claim 21 wherein the plurality of radiopaque markers are deposited into indentations in the exterior surface of the sheath.

27. The apparatus of claim 21 wherein the radiopaque markers consist of gold.

28. The apparatus of claim 21 wherein the proximal section of the sheath is coupled to a push wire.

29. The apparatus of claim 28 wherein the push wire spans a substantially greater length than the sheath.
